(19) United States
(12) Patent Application Publication
(10) Pub. No.: US 2011/0190739 A1
(43) Pub. Date: Aug. 4, 2011

(54) SERVO CONTROLLED DOCKING FORCE DEVICE FOR USE IN OPHTHALMIC APPLICATIONS

(75) Inventors: Rudolph W. Frey, Winter Park, FL (US); Steven E. Bott, Oviedo, FL (US)

(73) Assignee: LensAr, Inc.

(21) Appl. No.: 13/016,593

(22) Filed: Jan. 28, 2011

Related U.S. Application Data
(60) Provisional application No. 61/299,536, filed on Jan. 29, 2010.
(60) Provisional application No. 61/300,167, filed on Feb. 1, 2010.

(51) Int. Cl. A61F 9/009

(52) U.S. Cl. 606/4

(57) ABSTRACT

A laser treatment system that includes means for applying a laser beam to an eye of a patient and means for registering and immobilizing the eye.

This application claims the benefit of priority under 35 U.S.C. § 119(e)(1) of: 1) U.S. Provisional Application Ser. No. 61/299,536 titled Servo Controlled Docking Force Device for Use in Ophthalmic Applications, filed Jan. 29, 2010 and 2) U.S. Provisional Application Ser. No. 61/300,167 titled Servo Controlled Docking Force Device for Use in Ophthalmic Applications, filed Feb. 1, 2010, the entire contents of each of which is incorporated herein by reference.

### FIELD OF THE INVENTION

The present invention relates to a device to register and immobilize a patient's eye during a medical procedure, such as an ophthalmic procedure, and more particularly, the delivery of a treatment laser to the cornea of the eye.

### BACKGROUND

It is known to use a patient interface device (PID) with ophthalmic ultrashort pulse lasers used for cutting corneal or crystalline lens tissue of a patient's eye. The PID has two parts: a suction ring which is applied manually to the patient's eye and an upper docking part which is attached to a laser and then guided, via a joystick controlled 3-axis motion control system, to dock with the suction ring on the eye. The ultrashort pulse ophthalmic lasers provide visual or audible feedback to the surgeon about the magnitude of force being applied to the eye as the docking process is underway. The surgeon can manually adjust the position of the upper, docking part of the PID to try to apply sufficient force for the docking while avoiding excess force which could cause patient discomfort, compression of the anterior chamber, hemorrhaging or other trauma to the eye. However, the existing ultrashort pulse ophthalmic lasers do not have any automatic means to regulate the force applied to the eye.

The above described docking process using the PID is a manual operation and so it can be a time consuming process.

### BRIEF SUMMARY

One aspect of the present invention regards a laser treatment system that includes means for applying a laser beam to an eye of a patient and means for registering and immobilizing the eye.

A second aspect of the present invention regards a laser treatment system that includes a laser system that directs a laser beam to an eye of a patient and a patient interface device that engages the eye so as to register and immobilize the eye. The patient interface device includes a suction ring that is attached to the eye and a movable arm that engages the suction ring. The patient interface device further includes a transducer that measures a force generated by the patient interface device on the eye and a servo control system that receives a signal from the transducer that is representative of the measured force and the servo control system controls movement of the movable arm so that a force at a preset level is measured by the transducer.

A third aspect of the present invention regards a method of treating an eye, the method including directing a laser beam to an eye of a patient and engaging the eye with a patient interface device so as to register and immobilize the eye. The method further includes measuring a force generated by the patient interface device on the eye and controlling movement of the patient interface device so that a force at a preset level is subsequently measured.

One or more aspects of the present invention allow for a quick registration and immobilization of an eye.

### BRIEF DESCRIPTION OF THE DRAWING

The accompanying drawing, which is incorporated herein and constitutes part of this specification, and, together with the general description given above and the detailed description given below, serve to explain features of the present invention. In the drawings:

FIG. 1 is a side sectional and schematic view of an embodiment of a patient interface device in accordance with the present invention.

### DETAILED DESCRIPTION OF THE PREFERRED EMBODIMENTS

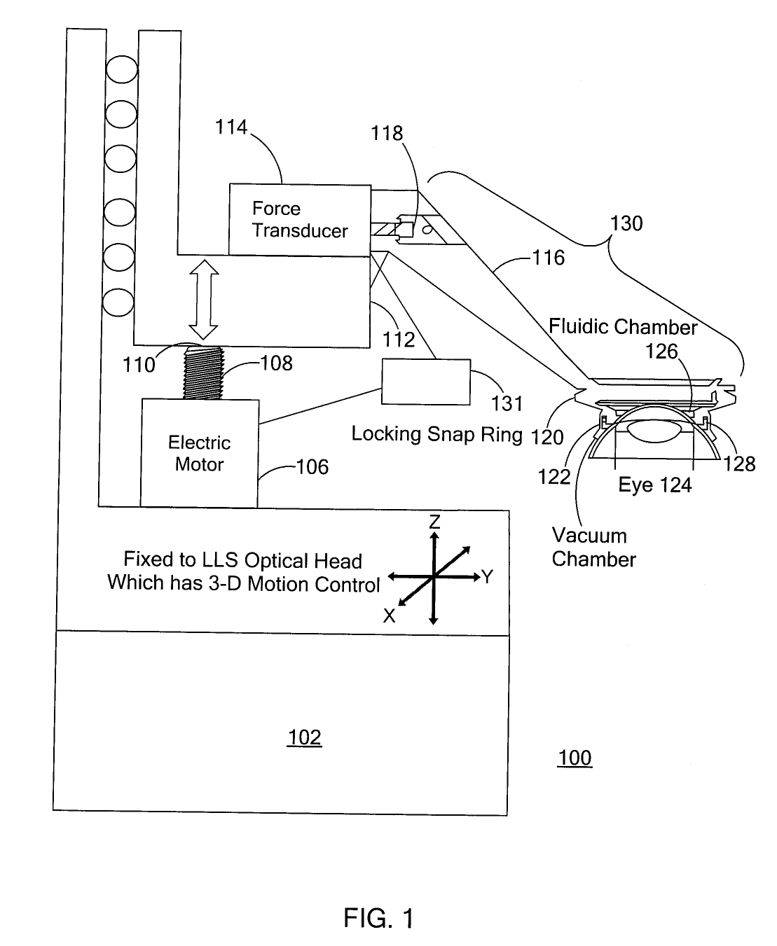

As schematically shown in FIG. 1, a laser treatment system 100 includes a laser system wherein only the optical head 102 is shown for convenience. The optical head moves in three dimensions in a well known manner via a three-dimensional motion control. Examples of possible laser systems that can be used are the laser systems described in U.S. patents applications Ser. Nos. 11/337,127; 12/217,285; 12/217,295; 12/509,412; 12/509,021; 12/509,211 and 12/509,454, the entire contents of each of which are incorporated herein by reference.

As shown in FIG. 1, an electric motor 106 is attached to the optical head 102. The electric motor 106 drives a translatable item, such as a screw 108, in the +z or −z directions. An end 110 of the screw 108 engages a translatable platform 112 that is able to move in the +z or −z directions. Thus, when the screw 108 engages platform 112 when moving in the +z direction, the platform 112 will also move in the +z direction. Similarly, when the screw 108 is engaged with platform 112 and moves in the −z direction, the platform 112 will also move in the −z direction.

As shown in FIG. 1, a force transducer 114 is attached to a top surface of the platform 112. The force transducer 114 is coupled to one end of an arm 116 via a mounting screw 118. The other end of the arm 116 has a locking snap ring 120.

Note that the mass of the platform 112, arm 116, force transducer 114 and locking snap ring 120 is approximately 1 Kg. This mass restricts the force on the eye to a maximum force of around 10N.

In operation, a suction ring 122 is applied manually to the patient's eye 124. As shown in FIG. 1, the suction ring 122 is centered on the eye 124 so that a chamber 126 encompasses the area of the eye 124 to be treated. In addition, a mounting wall 128 engages the surface of the eye 124. When the mounting wall 128 engages the eye 124, a vacuum is formed within the volume defined by the mounting wall 128 and the surface of the eye 124 encompassed by the mounting wall 128. Thus, the suction ring 122 is attached to the surface of the eye 124. In addition, a fluid is inserted in chamber 126.

Once the suction ring 122 is attached to the eye 124, a docking process is performed for the arm 116 that is separated from the suction ring 122. In particular, a surgeon moves a joystick (not shown) that controls a three axis motion servo-control system for controlling the motion of the optical head 102 in three dimensions. The surgeon moves the joystick to dock the locking snap ring 120 with the suction ring 122. When the locking ring 120 and suction ring 122 are docked, they constitute in combination a patient interface device 130. The servo controlled system restricts the force applied to the eye 124 in the event that the surgeon inadvertently moves the optical head 102 too quickly down onto the patient's eye 124.

As the patient interface device 130 is being docked to the eye 124, controlled by the surgeon using the joystick, the pressure applied to the eye 124 by the patient interface device 130 is monitored continuously by the force transducer 114 which measures the force applied between the arm 116 and the platform 112. A signal proportional to the measured force is sent from the transducer 114 to the servo control system 131 which in turn processes the signal so as to generate a control signal that is directed to the electric motor 106. The control signal controls the electric motor 106 so as to move the screw 108, platform 112 and arm 116 in the +z and −z directions. The control signal is such that if the measured force is above a preset level (i.e., eye is encountering too much pressure from the patient interface device 130), the screw 108, platform 112 and arm 116 are moved in the +z direction, lowering compression of the eye 124 by the patient interface device 130, until the preset level or the limit of travel of the screw 108 is reached. Similarly, if the measured force is below a preset level (i.e., eye is encountering too low a force from the patient interface device 130), the screw 108, platform 112 and arm 116 are moved in the −z direction until the preset level or the limit of travel of the screw 108 is reached. Note that the preset level is chosen such that the force applied to the eye is within a certain range so that it is 1) high enough to allow the upper docking part of the device to snap into the suction ring attached to the patient's eye and to maintain the eye in a fixed, stabile position and 2) but low enough that discomfort or trauma to the eye as well as undue compression of the eye's anterior chamber, is avoided. Depending on the exact design of the patient interface device 130, the amount of force required might be as low as one Newton or as high as 8 Newtons. Furthermore, the preset level may be in the range of 100 grams to 400 grams. The motor 106 moves the platform 112 and arm 116 in the +z direction if the measured force is above a preset level and moves the platform 112 and arm 116 in the −z direction if the force is below that level, thus maintaining the force on the eye near the preset level. Since the force on the eye 124 is maintained to an optimized preset level for every patient, the overall procedure is consistent—any tendency of the crystalline lens to move forward during the lasing procedure, in reaction to an over-compressed anterior chamber would be minimized.

In an alternative embodiment, the preset level may be varied during the docking procedure. For example, initial docking may be performed at a first preset level that ensures that the arm 116 and the locking snap ring 120 are firmly seated, (they snap together). Should the locking snap ring 120 be attached to the arm 116 during the docking process, then the first preset level is chosen so that the locking snap ring 120 snaps into engagement with the suction ring 128. After docking is achieved, the preset level is changed to a lower value that is sufficient to allow the patient interface device 130 to reach a z position that compresses the eye at an optimal level of force to maintain the stability of the eye and make it more comfortable for the patient.

Once the preset level is reached, docking is complete and the electric motor 106 is locked into position to prevent eye movement during the lasing procedure performed by the laser system 102. Thus, the patient interface device 130, which is used to register and immobilize the patient's eye 124 with respect to the laser, allows for efficient delivery of the treatment laser to the cornea and crystalline lens and allows for measurements of the position of the cornea and crystalline lens to be made through the patient interface device 130 and images of the lasing process to be provided to the user to monitor progress in the procedure.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A laser treatment system comprising:
means for applying a laser beam to an eye of a patient; and
means for registering and immobilizing said eye.

2. A laser treatment system comprising:
a laser system that directs a laser beam to an eye of a patient; and
a patient interface device that engages said eye so as to register and immobilize said eye, said patient interface device comprising:
a suction ring that is attached to said eye;
a movable arm that engages said suction ring;
a transducer that measures a force generated by said patient interface device on said eye; and
a servo control system that receives a signal from said transducer that is representative of said measured force and said servo control system controls movement of said movable arm so that a force at a preset level is measured by said transducer.

3. A method of treating an eye, the method comprising:
directing a laser beam to an eye of a patient;
engaging said eye with a patient interface device so as to register and immobilize said eye;
measuring a force generated by said patient interface device on said eye; and
controlling movement of said patient interface device so that a force at a preset level is subsequently measured.
